(19) United States
(12) Patent Application Publication
(10) Pub. No.: US 2018/0169030 A1
(43) Pub. Date: Jun. 21, 2018

(54) DRUG CARRIER AND METHOD OF FABRICATING THE SAME

(71) Applicant: National Tsing Hua University, Hsinchu City (TW)

(72) Inventors: Fan-Gang Tseng, Taipei City (TW); Chia-Wei Kang, Hsinchu City (TW); Shao-Hua Wang, New Taipei City (TW)

(73) Assignee: National Tsing Hua University, Hsinchu City (TW)

(21) Appl. No.: 15/458,384

(22) Filed: Mar. 14, 2017

(30) Foreign Application Priority Data
Dec. 20, 2016 (TW) 105142131

(51) Int. Cl. A61K 9/51; A61K 33/22

(52) U.S. Cl. CPC A61K 9/5161; A61K 9/5192; A61K 33/22

(57) ABSTRACT

A drug carrier and a method of fabricating the same are provided. The drug carrier is used to cover boron-containing drugs. The drug carrier includes a negatively charged polysaccharide inner core and a positively charged polysaccharide outer shell. The negatively charged polysaccharide inner core covers the boron-containing drugs. The positively charged polysaccharide outer shell covers a surface of the negatively charged polysaccharide inner core. The drug carrier can be delivered and gathered near tumor tissues. Also, a leakage of the boron-containing drugs in the delivery process is low, so as to reduce damage to normal cells.

### CROSS-REFERENCE TO RELATED APPLICATION

This application claims the priority benefit of Taiwan application serial no. 105142131, filed on Dec. 20, 2016. The entirety of the above-mentioned patent application is hereby incorporated by reference herein and made a part of this specification.

### BACKGROUND OF THE INVENTION

### Field of the Invention

The invention relates to a carrier and a method of fabricating the same, and particularly relates to a drug carrier and a method of fabricating the same.

### Description of Related Art

Cancer is one of the major causes of human death currently, and a common treatment method thereof is surgical treatment, chemical drug therapy, radiotherapy and the like, for example. The problem of traditional chemical drug therapy is that, the chemical drug input in human body not only attacks tumor cells, but also attacks normal cells, thereby causing great side effects on the human body.

In recent years, with advances in nanotechnology, studies have begun to use nanoparticles as drug carriers to deliver drugs. The advantage of using the nanoparticles as the drug carriers is that they are small in size, and can be easily absorbed by cells. Particularly, due to the enhanced permeability and retention effect (EPR effect), such drug carriers not only have an ability to carry and transport the drugs to a vicinity of the tumor tissues, but also the time for staying at the vicinity of the tumor tissues is longer, thereby increasing drug selectivity and reducing the side effects.

### SUMMARY OF THE INVENTION

The invention provides a drug carrier, which can cover boron-containing drugs to be delivered and gathered near the tumor tissues. Also, a leakage of the boron-containing drugs in the delivery process is low, so as to reduce the damage to the normal cells.

The embodiment of the invention provides a drug carrier used to cover boron-containing drugs. The drug carrier includes a negatively charged polysaccharide inner core and a positively charged polysaccharide outer shell. The negatively charged polysaccharide inner core covers the boron-containing drugs. The positively charged polysaccharide outer shell covers a surface of the negatively charged polysaccharide inner core.

According to some embodiments of the invention, a component of the negatively charged polysaccharide inner core includes alginate, gelatin, or a combination thereof.

According to some embodiments of the invention, a component of the positively charged polysaccharide outer shell includes chitosan.

According to some embodiments of the invention, the boron-containing drugs include boric acid, boronophenylalanine (BPA), sodium borocaptate (BSH), or a combination thereof.

According to some embodiments of the invention, a particle size range of the drug carrier is between 150 nanometers and 250 nanometers.

According to some embodiments of the invention, a loading efficiency range of the drug carriers to the boron-containing drugs is between 15% and 25%.

The embodiment of the invention provides a method of fabricating the drug carrier used to cover boron-containing drugs including the following steps. The boron-containing drugs are mixed with a negatively charged polysaccharide solution to form a mixed solution. An electrospray process is performed. The mixed solution is electrosprayed into an aqueous solution to form a negatively charged polysaccharide inner core in the aqueous solution, wherein the negatively charged polysaccharide inner core covers the boron-containing drugs. The negatively charged polysaccharide inner core is immersed in a positively charged polysaccharide solution such that a surface of the negatively charged polysaccharide inner core is covered with a positively charged polysaccharide outer shell.

According to some embodiments of the invention, a component of the negatively charged polysaccharide solution includes alginate, gelatin, or a combination thereof.

According to some embodiments of the invention, a component of the positively charged polysaccharide solution includes chitosan.

According to some embodiments of the invention, the boron-containing drugs include boric acid, boronophenylalanine, sodium borocaptate, or a combination thereof.

According to some embodiments of the invention, the aqueous solution includes a strontium chloride aqueous solution, a calcium chloride aqueous solution, or a combination thereof.

According to some embodiments of the invention, an applied voltage of the electrospray process is between 13 kV and 15 kV.

According to some embodiments of the invention, a flow rate range of the mixed solution in the electrospray process is between 20 microliters/hour and 50 microliters/hour.

According to some embodiments of the invention, a time for immersing the negatively charged polysaccharide inner core in the positively charged polysaccharide solution is at least 12 hours or more.

According to some embodiments of the invention, a temperature for immersing the negatively charged polysaccharide inner core in the positively charged polysaccharide solution is room temperature.

According to some embodiments of the invention, a particle size range of the drug carrier is between 150 nanometers and 250 nanometers.

According to some embodiments of the invention, the positively charged polysaccharide outer shell covers the surface of the negatively charged polysaccharide inner core by electrostatic attraction.

According to some embodiments of the invention, a loading efficiency range of the drug carriers to the boron-containing drugs is between 15% and 25%.

Based on the above, the drug carrier of the invention can be used to cover the boron-containing drugs. The drug carrier includes the negatively charged polysaccharide inner core and the positively charged polysaccharide outer shell, wherein the negatively charged polysaccharide inner core covers the boron-containing drugs, and the positively charged polysaccharide outer shell covers the surface of the negatively charged polysaccharide inner core. The drug carrier of the invention is prepared by electrospraying. By controlling the parameters, such as the flow rate of fluid, the applied voltage, the particle size of the drug carrier can be adjusted to the size which is suitable for delivering and gathering near the tumor tissues so as to increase the treatment effect. On the other hand, since the positively charged polysaccharide outer shell covers the surface of the negatively charged polysaccharide inner core, the leakage of the boron-containing drugs in the delivery process can be decreased, thereby the damage to the normal cells can be reduced.

In order to make the aforementioned features and advantages of the disclosure more comprehensible, embodiments accompanied with figures are described in detail below.

### BRIEF DESCRIPTION OF THE DRAWINGS

The accompanying drawings are included to provide a further understanding of the invention, and are incorporated in and constitute a part of this specification. The drawings illustrate embodiments of the invention and, together with the description, serve to explain the principles of the invention.

FIG. 1 is a schematic structure diagram illustrating a drug carrier according to some embodiments of the invention.

FIG. 2 is a schematic flow chart illustrating a method of fabricating the drug carrier according to some embodiments of the invention.

FIG. 3A to FIG. 3C are schematic diagrams illustrating a manufacturing process of the drug carrier according to some embodiments of the invention.

FIG. 4A is a diagram illustrating the relationship between particle size of the drug carrier and applied voltage according to various embodiments of the invention.

FIG. 4B is a diagram illustrating the relationship between particle size of the drug carrier and liquid concentration/applied voltage according to various embodiments of the invention.

FIG. 5A is a scanning electron microscope (SEM) diagram of the drug carrier of some embodiments of the invention.

FIG. 5B and FIG. 5C are partial enlarged views of FIG. 5A.

FIG. 6 is a calibration curve diagram illustrating a boron content of the drug carrier covering boric acid according to some embodiments of the invention.

FIG. 7 illustrates cell toxicity results of boric acid containing different concentrations of boron-10, the drug carrier, and the drug carrier covering boric acid according to various embodiments of the invention.

FIG. 8 is a schematic diagram illustrating thermal neutron irradiation according to some embodiments of the invention.

FIG. 9 illustrates cell viability results after thermal neutron irradiation of boric acid, the drug carrier, and the drug carrier covering boric acid according to the embodiments of the invention.

### DESCRIPTION OF THE EMBODIMENTS

In the following detailed description, for purposes of explanation, numerous specific details are set forth in order to provide a thorough understanding of the disclosed embodiments. It will be apparent, however, that one or more embodiments may be practiced without these specific details. In other instances, well-known structures and devices are schematically shown in order to simplify the drawing.

The drug carrier is mainly to cover the drugs therein, and then treatment is performed by controlling the time of drug release. The use of such drug carrier has less side effects compared with the traditional chemical drug therapy. However, in a process of drug delivery, the leakage of the drugs from the drug carrier may still damage other normal cells. Thus, the drug carrier provided by the embodiments of the invention can make the drugs be delivered and gathered near the tumor tissues. At the same time, the leakage of the drugs in the delivery process can be reduced.

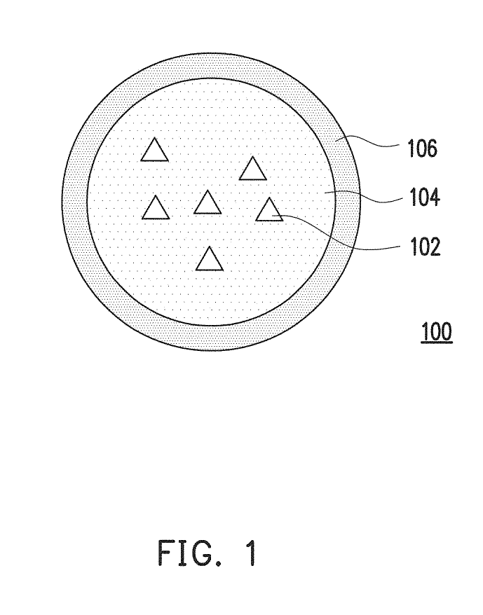

FIG. 1 is a schematic structure diagram illustrating a drug carrier according to some embodiments of the invention.

Referring to FIG. 1, a drug carrier 100 of the embodiment may be used to cover boron-containing drugs 102. The drug carrier 100 may include a negatively charged polysaccharide inner core 104 and a positively charged polysaccharide outer shell 106. The negatively charged polysaccharide inner core 104 covers the boron-containing drugs 102, and the positively charged polysaccharide outer shell 106 covers a surface of the negatively charged polysaccharide inner core 104.

<Negatively Charged Polysaccharide Inner Core>

A component of the negatively charged polysaccharide inner core 104 includes alginate, gelatin, or a combination thereof, for example. In some embodiments, a molecular weight range of the negatively charged polysaccharide inner core 104 is between 10000 Dalton (Da) and 600000 Da, for example. A particle size range of the negatively charged polysaccharide inner core 104 is between 150 nanometers and 250 nanometers, for example. A method of preparing the negatively charged polysaccharide inner core 104 is an electrospray method, for example. The electrospray method will be described in more detail below. In a particular embodiment, the component of the negatively charged polysaccharide inner core 104 is alginate, for example. Alginate is a colloid formed by cross-linking sodium alginate molecules with divalent metal cations, for example. The divalent metal cations include calcium ions, strontium ions, and the like, for example. However, the invention is not limited thereto.

The structure of alginate is as follows:

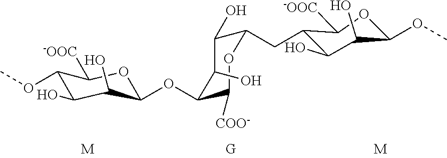

<Positively Charged Polysaccharide Outer Shell>

A component of the positively charged polysaccharide outer shell 106 includes chitosan, for example. In some embodiments, a molecular weight of the positively charged polysaccharide outer shell 106 is between 300 Da and 310000 Da, for example. A thickness of the positively charged polysaccharide outer shell 106 is about 10 nanometers, for example. In some embodiments, a loading efficiency of the positively charged polysaccharide outer shell 106 covering the surface of the negatively charged polysaccharide inner core 104 is 15% or more, and a range thereof is between 15% and 25%, for example. In some exemplary embodiments, the positively charged polysaccharide outer shell 106 does not have holes. That is to say the positively charged polysaccharide outer shell 106 completely covers the surface of the negatively charged polysaccharide inner core 104 such that the negatively charged polysaccharide inner core 104 is not exposed. In the embodiment, since the positively charged polysaccharide outer shell 106 completely covers the surface of the negatively charged polysaccharide inner core 104, the chance of the leakage of the boron-containing drugs 102 covered in the negatively charged polysaccharide inner core 104 from the drug carrier 100 can be reduced. However, the invention is not limited thereto. In some embodiments, the positively charged polysaccharide outer shell 106 covers the surface of the negatively charged polysaccharide inner core 104 by electrostatic attraction, for example.

The structure of chitosan is as follows:

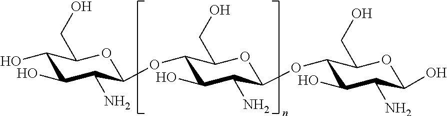

<Boron-Containing Drugs>

The boron-containing drugs 102 include boric acid, boronophenylalanine (BPA), sodium borocaptate (BSH), or a combination thereof, for example. In some embodiments, a concentration range of the boron-containing drugs 102 is between 2 wt % and 5 wt %, for example. The boron-containing drugs 102 are evenly distributed in the negatively charged polysaccharide inner core 104, for example. A loading efficiency of the drug carriers 100 to the boron-containing drugs 102 may be 15% or more, and a range thereof is between 15% and 25%, for example.

The structure of boric acid is as follows:

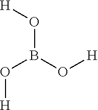

The structure of boronophenylalanine is as follows:

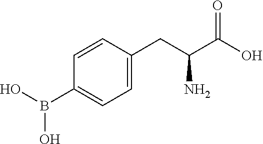 [SMILES: N[C@@H](Cc1ccc(B(O)O)cc1)C(=O)O]

The structure of sodium borocaptate is as follows:

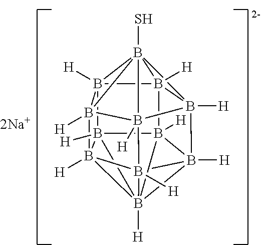

<Drug Carrier>

A particle size range of the drug carrier 100 may be related to the target to be treated. In some embodiments, the particle size range of the drug carrier 100 is between 150 nanometers and 250 nanometers, for example. In some other embodiments, the particle size range of the drug carrier 100 is between 200 nanometers and 250 nanometers, for example. In some embodiments, the positively charged polysaccharide outer shell 106 of the drug carrier 100 can be selectively modified with a substance which can increase selectivity thereon, such as grafted antibody, so as to improve drug selectivity of the overall drug carrier.

It should be noted that the boron-containing drugs 102 covered in the drug carrier 100 of the invention can be used in boron neutron capture therapy (BNCT). Specifically, the BNCT is to make the drugs containing isotope boron-10 (boron-10, B¹⁰) selectively gather near the tumor tissues, and thermal neutrons or epithermal neutrons are used to irradiate the tumor tissues in vitro to kill cancer cells. More specifically, the boron-10 will split into ⁷Li and ⁴He after thermal neutron or epithermal neutron irradiation. The two radiation particles have a quite large linear energy transfer (LET), and distances of energy release thereof are 5 micrometers and 9 micrometers respectively, which is approximately a diameter of one mammalian cell (about 10 micrometers). That is to say a radiation range of the two radiation particles is limited to one nucleus and its adjacent cells, and the high linear energy transfer thereof is fully released into the cell in a short distance. Thus, if the particle size of the drug carrier of the invention is further adjusted by controlling process parameters, so that it can be delivered and gathered near the tumor tissues. When it is used in the BNCT, it can not only cause DNA double strand break (DSB) of the cancer cells effectively to kill the cancer cells, but also reduce the damage to the normal cells.

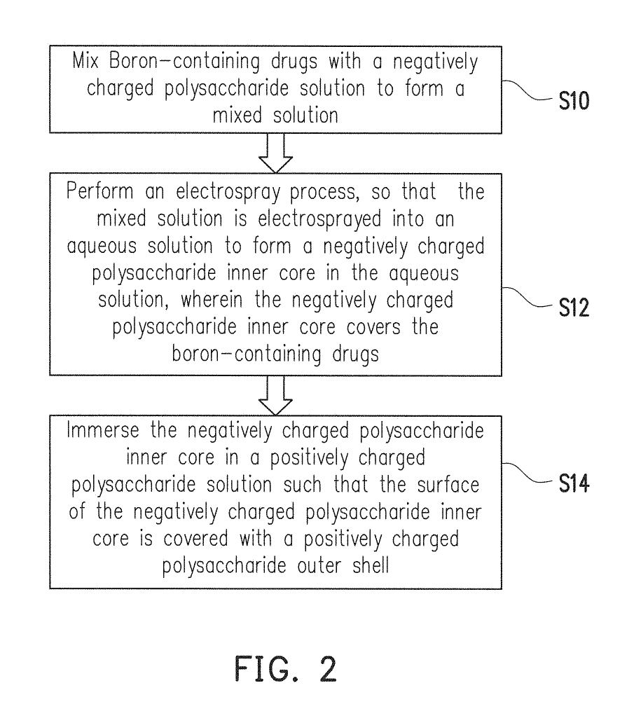

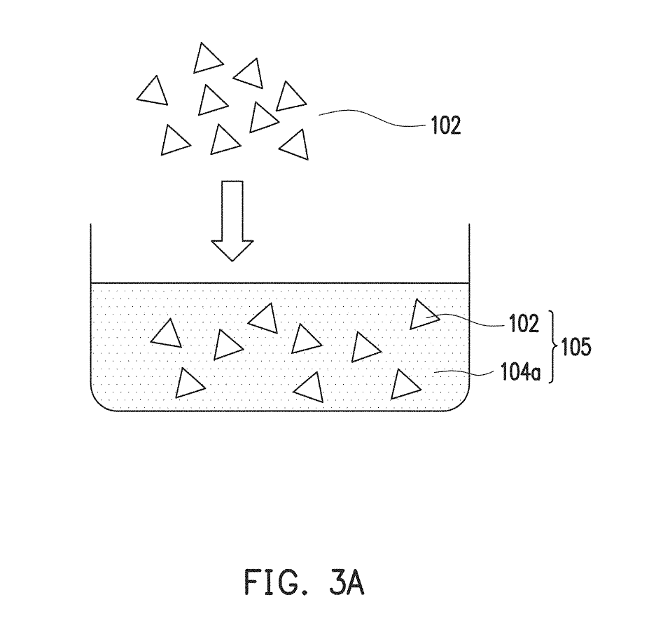

FIG. 2 is a schematic flow chart illustrating a method of fabricating the drug carrier according to some embodiments of the invention. FIG. 3A to FIG. 3C are schematic diagrams illustrating a manufacturing process of the drug carrier according to some embodiments of the invention.

Referring to FIG. 2 and FIG. 3A to FIG. 3C, the method of fabricating the drug carrier 100 of the invention includes the following steps. The boron-containing drugs 102 are mixed with a negatively charged polysaccharide solution 104a to form a mixed solution 105 (Step S10). An electrospray process is performed. The mixed solution 105 is electrosprayed into an aqueous solution 107 to form the negatively charged polysaccharide inner core 104 in the aqueous solution 107, wherein the negatively charged polysaccharide inner core 104 covers the boron-containing drugs 102 (Step S12). The negatively charged polysaccharide inner core 104 is immersed in a positively charged polysaccharide solution 106a such that the surface of the negatively charged polysaccharide inner core 104 is covered with the positively charged polysaccharide outer shell 106 (Step S14). The above steps will be described in more detail below.

<Step S10>

Referring to FIG. 2 and FIG. 3A, first, Step S10 is performed. The boron-containing drugs 102 are mixed with the negatively charged polysaccharide solution 104a to form the mixed solution 105. In some embodiments, the boron-containing drugs 102 are boric acid, for example. The concentration of the boron-containing drugs 102 is between 2 wt % and 5 wt %, for example. In some embodiments, the negatively charged polysaccharide solution 104a is an alginate solution, for example. The concentration of the negatively charged polysaccharide solution 104a is between 1 wt % and 2 wt %, for example. The mixing method is, for example, the boron-containing drugs 102 and the negatively charged polysaccharide solution 104a are prepared respectively. Then, the boron-containing drugs 102 are directly added into the negatively charged polysaccharide solution 104a. After homogeneous mixing, the mixed solution 105 can be obtained. In some embodiments, the temperature for performing mixing is room temperature, for example.

<Step S12>

Referring to FIG. 2 and FIG. 3B, Step S12 is performed. The electrospray process is performed. The mixed solution 105 is electrosprayed into the aqueous solution 107 to form the negatively charged polysaccharide inner core 104 in the aqueous solution 107, wherein the negatively charged polysaccharide inner core 104 covers the boron-containing drugs 102. In some embodiments, in the electrospray process, a flow rate range of the mixed solution 105 in a syringe 110 is between 20 microliters/hour and 50 microliters/hour, for example, and an applied voltage range is between 13 kV and 15 kV, for example. In some embodiments, the aqueous solution 107 is a salt aqueous solution, for example. For instance, the aqueous solution 107 is a divalent metal cation aqueous solution, for example, such as including strontium chloride (SrCl₂) aqueous solution, calcium chloride (CaCl₂) aqueous solution, or a combination thereof. A concentration range of the aqueous solution 107 is between 0.1 M±5%, for example.

Specifically, the mixed solution 105 prepared in Step S10 is directly electrosprayed into the aqueous solution 107 using the electrospray process. More particularly, in the electrospray process, a high voltage is applied to give an electric field, so that a surface of a liquid bead of the mixed solution 105 ejected from the syringe 110 is charged. As the charge density of the surface of the liquid bead is gradually increased and concentrated at a top of the liquid bead, a liquid surface will be stretched and deformed to form a cone, which is called “Taylor cone”. When the applied voltage is continuously increased to exceed the threshold voltage of surface tension of the solution, the mixed solution 105 will be broken to form droplets to be emitted from the tip of the Taylor cone. Then, the droplets will repeat the step of self-cleavage to form a plurality of negatively charged polysaccharide particles 105a covering the boron-containing drugs 102 therein. In this stage, the particle size of the negatively charged polysaccharide particles 105a can be controlled by controlling the applied voltage and the flow rate of the mixed solution 105 pushed by the syringe 110. Then, the formed negatively charged polysaccharide particles 105a are directly placed in the aqueous solution 107 to be collected, so as to form the negatively charged polysaccharide inner cores 104 covering the boron-containing drugs 102 therein.

<Step S14>

Referring to FIG. 2 and FIG. 3C, Step S14 is performed. The negatively charged polysaccharide inner core 104 is immersed in the positively charged polysaccharide solution 106a such that the surface of the negatively charged polysaccharide inner core 104 is covered with the positively charged polysaccharide outer shell 106. In some embodiments, the positively charged polysaccharide solution 106a is a chitosan solution, for example. A concentration range of the positively charged polysaccharide solution 106a is between 1 wt % and 2 wt %, for example. In some embodiments, an immersion time can be adjusted according to the thickness of the positively charged polysaccharide outer shell 106 to be formed. The time for immersing the negatively charged polysaccharide inner core 104 in the positively charged polysaccharide solution 106a is between 30 minutes and 24 hours, for example. In some embodiments, the time for immersing the negatively charged polysaccharide inner core 104 in the positively charged polysaccharide solution 106a is 12 hours or more, for example. In some embodiments, the temperature for immersing the negatively charged polysaccharide inner core 104 in the positively charged polysaccharide solution 106a is room temperature, for example.

The invention will be described in more detail with reference to specific experimental examples.

### Experimental Example

First, 0.1 g of alginate and 0.07 g of alginate were respectively added into 10 ml of 1X phosphate buffered saline (PBS), and after heating at 60° C. using a hot plate and stirring for 1 hour to be completely dissolved, they were placed at room temperature to form 1 wt % of alginate solution and 0.7 wt % of alginate solution. Next, 3 wt % of boric acid was directly added into 1 wt % of alginate solution or 0.7 wt % of alginate solution and evenly mixed to obtain a mixed solution.

Before proceeding to the subsequent electrospray step, 0.1 M of strontium chloride aqueous solution and 1 wt % of chitosan solution can be prepared first for use in subsequent steps. The preparation of 0.1 M of strontium chloride aqueous solution was, for example, to add 4.381 g of strontium chloride into 500 ml of deionized water and stir to be completely dissolved. Then, 0.1 M of strontium chloride aqueous solution was placed in an aluminum collector to be used as a collector for the subsequent electrospray step. The preparation of 1% of chitosan solution was, for example, to add 1% (W/V) of chitosan into 1% (W/V) of acetic acid solution and stirred with a magnet to be completely dissolved to form 1% of chitosan solution.

250 μl of syringe was used to aspirate 250 μl of mixed solution. The syringe was pushed using a syringe pump. The flow rate of the mixed solution is set to 30 μl/hour and 40 μl/hour respectively. Then, a high voltage electric field was externally connected to provide stable voltage (14 kV, 14.5 kV, and 15 kV) such that the liquid surface of the mixed solution pushed out from the syringe formed the Taylor cone. Then, 200 μl of mixed solution was electrosprayed, and alginate particles sprayed out from a syringe needle were collected in the aluminum collector with 0.1 M of strontium chloride aqueous solution. Then, the strontium chloride aqueous solution containing the alginate particles was collected using a dropper, and then the supernatant was removed after centrifugation (14000 rpm, 30 minutes).

1% of chitosan solution was added into the remaining lower-layer liquid. The alginate particles were scattered after ultrasonic vibration, so that chitosan can evenly cover the surface of the alginate particles. After standing for 12 hours, the supernatant was removed after centrifugation (14000 rpm, 30 minutes). The deionized water was added, and the particles were scattered after ultrasonic vibration. The drug carrier of the embodiment was obtained.

After the drug carrier was prepared, the morphology and particle size of the drug carrier were observed. Also, boric acid loading efficiency, cell toxicity, and an ability of boron neutron capture therapy for cells of the drug carrier were further tested.

<Observation of Morphology and Particle Size>

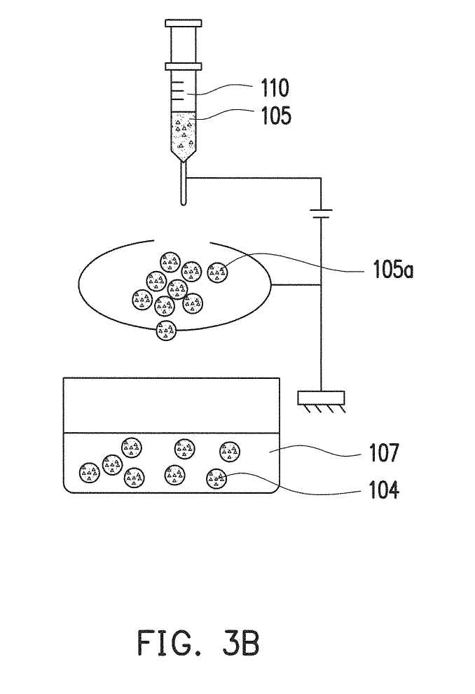

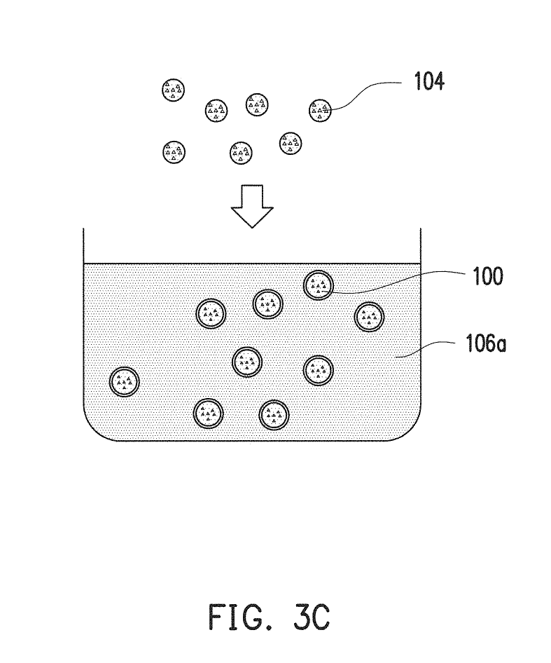

FIG. 4A is a diagram illustrating the relationship between particle size of the drug carrier and applied voltage according to various embodiments of the invention. FIG. 4B is a diagram illustrating the relationship between particle size of the drug carrier and liquid concentration/applied voltage according to various embodiments of the invention. FIG. 5A is a scanning electron microscope (SEM) diagram of the drug carrier of some embodiments of the invention. FIG. 5B and FIG. 5C are partial enlarged views of FIG. 5A.

In the embodiment, the particle size of the drug carrier prepared by applying different voltages (9 kV, 10 kV, 11 kV, 12 kV, 13 kV, 14 kV, 14.5 kV, 15 kV) at the same flow rate (30 μl/hour) was observed using a zetasizer and a scanning electron microscope. Additionally, the particle size of the drug carrier prepared by applying different voltages (14.5 kV, 15 kV) at different concentrations (0.7 wt %, 1 wt %) of alginate solution was observed.

From FIG. 4A, a threshold voltage for forming the Taylor cone is about 14.5 kV. Specifically, at the same flow rate (30 μl/hour), before the applied voltage is lower than 14.5 kV, the particle size of the drug carrier will decrease with the increase of the applied voltage. When the applied voltage is 14.5 kV, the particle size of the drug carrier can achieve a minimum value. Then, when the applied voltage is 15 kV (more than 14.5 kV), the applied voltage at this time is over the threshold voltage for forming the Taylor cone. Thus, the particle size of the drug carrier will also increase with the increase of the applied voltage. It should be noted that at the same applied voltage, when the flow rate is increased to 40 μl/hour, the particle size of the drug carrier will increase (not shown). That is to say, at the same applied voltage, the particle size of the drug carrier when the flow rate is 40 μl/hour will larger than the particle size of the drug carrier when the flow rate is 30 μl/hour. In other words, at the same applied voltage, the particle size of the drug carrier will also increase with the increase of the flow rate.

From FIG. 4B, at the same applied voltage, the concentration of the alginate solution has no significant effect on the particle size of the drug carrier.

Referring to FIG. 5A to FIG. 5C, in the embodiment, the drug carrier was prepared under a condition that the flow rate was 30 μl/hour and the applied voltage was 14.5 kV, and the prepared drug carrier was freeze-dried and then observed by SEM. FIG. 5B and FIG. 5 are partial enlarged views of a particle a and a particle b in FIG. 5A respectively. A particle size d1 of the particle a is about 198 nanometers. A particle size d2 of the particle b is about 205 nanometers. The results are consistent with the results of FIG. 4A. That is, the particle size of the drug carrier prepared under this condition is about 200 nanometers.

On the other hand, a zeta potential of the surface of the drug carrier was measured using the zetasizer, so as to confirm whether the chitosan outer shell covers the surface of the alginate inner core by electrostatic attraction to form the drug carrier. According to the results of Table 1 below, before the alginate particles have not been covered with the chitosan outer shell, since alginate itself is negatively charged, the measured zeta potential is negative. That is, the surface of the alginate particles is negatively charged. After the alginate particles have been covered with the chitosan outer shell, since chitosan itself is positively charged, the measured zeta potential is positive. That is, the surface of the chitosan outer shell is positively charged. That is to say, the structure of the drug carrier comprises the negatively charged alginate inner core and the positively charged chitosan outer shell.

TABLE 1

| | Zeta potential (mV) |
|---|---|
| Alginate particles | −1.17 |
| Surface of alginate particles covered with | +8.37 |
| chitosan outer shell | |

<Boric Acid Loading Efficiency Test>

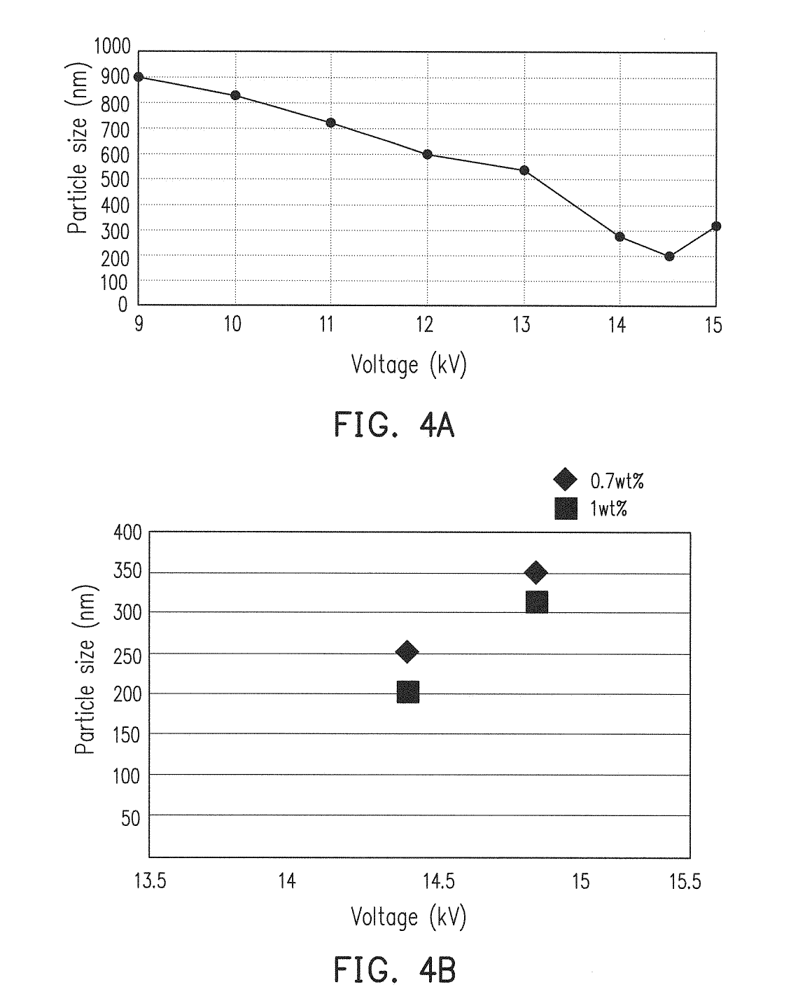

FIG. 6 is a calibration curve diagram illustrating a boron content of the drug carrier covering boric acid according to some embodiments of the invention.

The content of boron-10 in the drug carrier is quantified using an inductively coupled plasma-mass spectrometer (ICP-MASS) to estimate the boric acid loading efficiency thereof. The calculation formula is as follows:

Loading efficiency(%)=(Total amount of drugs−Free drugs in the supernatant (remaining drugs))/Total amount of drugs

Then, as shown in FIG. 6, the calibration curve of the boron content is depicted first. The horizontal axis (x-axis) is the concentration of boron-10 (ppb). The vertical axis (y-axis) is the intensity count value (counts/sec; CPS). The calibration curve formula is y=0.3676*x+1.0100. The coefficient of correlation (R) is 0.9976. The detection limit (DL) is 14.28 ppb. The background equivalent concentration (BEC) is 2.748 ppb. Then, according to the calibration curve, as the molecular weight of the chitosan outer shell is 190 kDa, CPS=55.56 when the drug carrier is diluted 20 times. At this time, the actual content of boron-10 is 3 ppm, the total amount of boron-10 is 75 ppm, and the loading efficiency, which is 3.9%, can be obtained by substituting into the formula. As the molecular weight of the chitosan outer shell is 19 kDa, CPS=210.103 when the drug carrier is diluted 20 times. At this time, the actual content of boron-10 is 11.4 ppm, the total amount of boron-10 is 75 ppm, and the loading efficiency, which is 15.2%, can be obtained by substituting into the formula. The subsequent cell toxicity test is based on the chitosan with the molecular weight of 19 kDa as the outer shell of the drug carrier.

<Cell Toxicity Test>

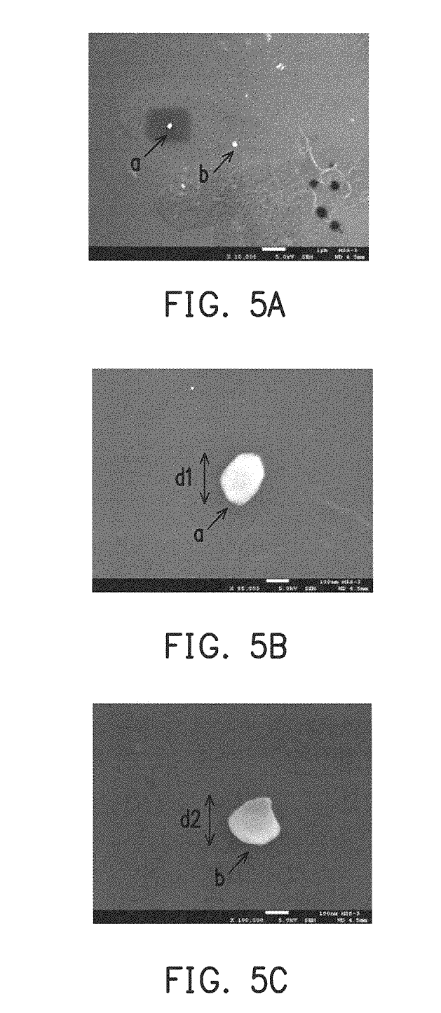

FIG. 7 illustrates cell toxicity results of boric acid containing different concentrations of boron-10, the drug carrier, and the drug carrier covering boric acid according to various embodiments of the invention.

Boric acids with different concentrations of boron-10 (15 ppm, 30 ppm, 75 ppm, 187.5 ppm, 375 ppm) are prepared respectively and the drug carrier covering boric acid prepared in the above embodiment are dissolved in cell culture medium. Then, hepatoma cell line (HepG2) was cultured therein. After 24 hours, cell toxicity analysis kit (Wst-1 assay) was used. After reaction for 4 hours, absorbance values were measured to test cell viability.

As shown in FIG. 7, the horizontal axis represents groups in different conditions. Group A to Group H were prepared according to Table 2 below. The vertical axis is cell viability (%). According to the results of cell viability of each group, it can be found that the cell toxicity of 15 ppm concentration of boron-10 (Group B) and the cell toxicity of 30 ppm concentration of the boron-10 (Group C) are low. 75 ppm concentration of boron-10 (Group D) has a considerable degree of cell toxicity (cell viability is about 56%). 75 ppm or more concentration of boron-10 (Group E and Group F) have great cell toxicity (cell viability is about 20% or less). It should be note that, compared with 75 ppm concentration of boron-10 (Group D), the cell viability of boric acid having 75 ppm concentration of boron-10 covered with the drug carrier (Group G) may be up to 85%. That is to say, boric acid covered with the drug carrier can significantly reduce the cell toxicity. Also, the cell viability of the drug carrier only (Group H) can be up to 90%. That is to say, the cell toxicity of the drug carrier only is low. According to the above results, it can be concluded that the drug carrier of the invention has a high degree of cell compatibility (namely, low cell toxicity), and it can cover boric acid to reduce the cell toxicity of boric acid.

TABLE 2

| Group | A | B | C | D | E | F | G | H |
|---|---|---|---|---|---|---|---|---|
| Boron-10 | − | 15 | 30 | 75 | 187.5 | 375 | 75 | − |
| concentration | | | | | | | | |
| (ppm) | | | | | | | | |
| Drug carrier | − | − | − | − | − | − | + | + |

<Ability of Boron Neutron Capture Therapy for Cells Test>

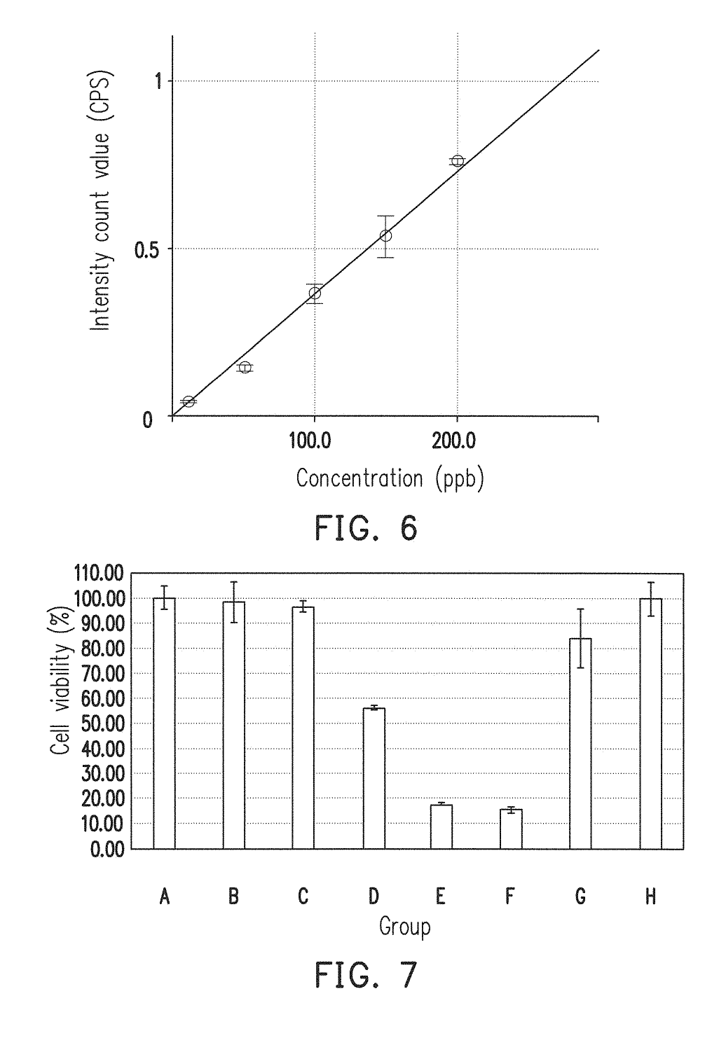

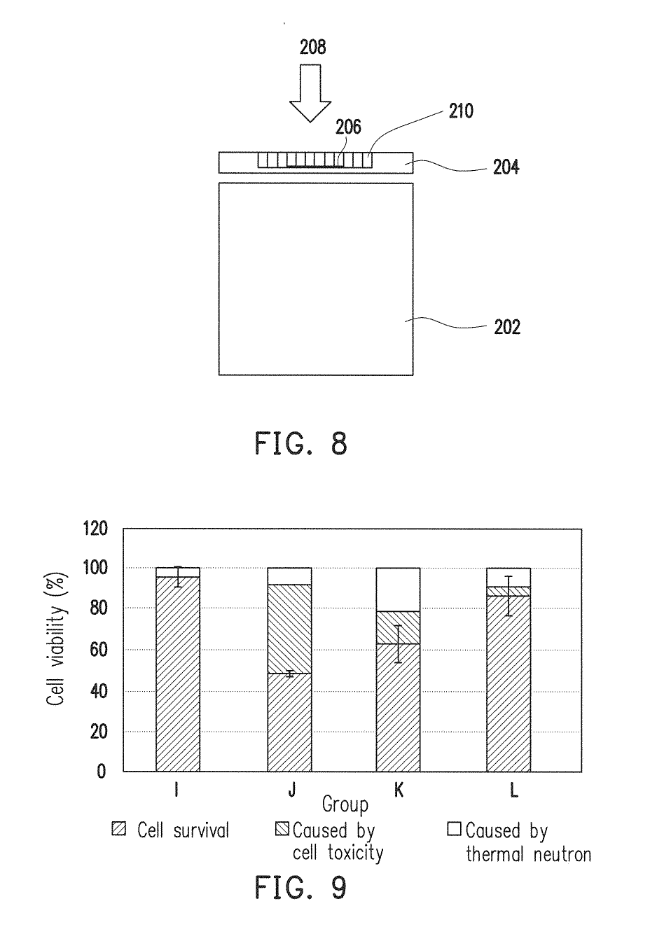

FIG. 8 is a schematic diagram illustrating thermal neutron irradiation according to some embodiments of the invention. FIG. 9 illustrates cell viability results after thermal neutron irradiation of boric acid, the drug carrier, and the drug carrier covering boric acid according to the embodiments of the invention.

The drug carrier prepared in the above embodiments applied to the boron neutron capture therapy for cells was tested. The test method is described in detail below. Referring to FIG. 8, Tsing Hua Open-pool Reactor (THOR) 200 was used. An acrylic plate 204 was used as a carrier of 96-well plate 210. Hepatoma cells 206 were placed in the 96-well plate. Thermal neutrons 208 were used to irradiate the 96-well plate 210. An acrylic cube 202 was placed behind the carrier to slow down the thermal neutrons 208, so as to shorten the irradiation time.

As shown in FIG. 9, the horizontal axis represents groups. Group I represents culture medium. Group J represents a boric acid solution containing 75 ppm of boron-10. Group K represents the drug carrier covering boric acid containing 75 ppm of boron-10. Group L represents the drug carrier without covering boric acid. The vertical axis is cell number percentage (%). The thermal neutron irradiation time was 27.67 minutes. After the thermal neutron irradiation, changes in cell numbers of each group were tested using the cell toxicity analysis kit (Wst-1 assay) after 24 hours.

As shown in FIG. 9, the cell number of Group I without irradiation of thermal neutron was used as cell percentage of the control group. According to the results of changes in cell numbers of Group I, it can be found that there is no significant difference between the cell number of Group I with or without irradiation of thermal neutron. That is to say, the damage to cells by irradiation of thermal neutron only is little. According to the results of changes in cell numbers of Group J, the cell viability obtained after irradiation of thermal neutron for 24 hours is remaining 45%, wherein 40% in 55% of mortality rate is caused by toxicity of boric acid, and only 15% is caused by irradiation of thermal neutron. According to the results of changes in cell numbers of Group K, the cell viability obtained after irradiation of thermal neutron for 24 hours is 60%, wherein 15% in 40% of mortality rate is caused by drug carrier, and 25% is caused by irradiation of thermal neutron. It can be seen that, boric acid covered with the drug carrier can reduce the toxicity of boric acid to the cells and increase the cell mortality rate caused by the reaction between thermal neutrons and boron-10. According to the results of changes in cell numbers of Group L, it can be found that changes in cell numbers of the drug carrier without covering boric acid (Group L) and the culture medium only (Group I) is less than 10% after irradiation of thermal neutron. It represents that the cell toxicity of the drug carrier without covering boric acid is quite low.

In summary, the drug carrier of the invention can be used to cover the boron-containing drugs. The drug carrier includes the negatively charged polysaccharide inner core and the positively charged polysaccharide outer shell, wherein the negatively charged polysaccharide inner core covers the boron-containing drugs, and the positively charged polysaccharide outer shell covers the surface of the negatively charged polysaccharide inner core. The drug carrier of the invention is prepared by electrospraying. By controlling the parameters, such as the flow rate of fluid, the applied voltage, the particle size of the drug carrier can be adjusted to the size which is suitable for delivering and gathering near the tumor tissues so as to increase the treatment effect. On the other hand, since the positively charged polysaccharide outer shell covers the surface of the negatively charged polysaccharide inner core, the leakage of the boron-containing drugs in the delivery process can be decreased, thereby the damage to the normal cells can be reduced.

Although the invention has been described with reference to the above embodiments, it will be apparent to one of ordinary skill in the art that modifications to the described embodiments may be made without departing from the spirit of the invention. Accordingly, the scope of the invention is defined by the attached claims not by the above detailed descriptions.

1. A drug carrier used to cover boron-containing drugs, comprising:
a negatively charged polysaccharide inner core, covering the boron-containing drugs; and
a positively charged polysaccharide outer shell, covering a surface of the negatively charged polysaccharide inner core.

2. The drug carrier according to claim 1, wherein a component of the negatively charged polysaccharide inner core comprises alginate, gelatin, or a combination thereof.

3. The drug carrier according to claim 1, wherein a component of the positively charged polysaccharide outer shell comprises chitosan.

4. The drug carrier according to claim 1, wherein the boron-containing drugs comprise boric acid, boronophenylalanine (BPA), sodium borocaptate (BSH), or a combination thereof.

5. The drug carrier according to claim 1, wherein a particle size range of the drug carrier is between 150 nanometers and 250 nanometers.

6. The drug carrier according to claim 1, wherein a loading efficiency range of the drug carriers to the boron-containing drugs is between 15% and 25%.

7. A method of fabricating a drug carrier used to cover boron-containing drugs, comprising:
mixing the boron-containing drugs with a negatively charged polysaccharide solution to form a mixed solution;
performing an electrospray process, electrospraying the mixed solution into an aqueous solution to form a negatively charged polysaccharide inner core in the aqueous solution, wherein the negatively charged polysaccharide inner core covers the boron-containing drugs; and
immersing the negatively charged polysaccharide inner core in a positively charged polysaccharide solution such that a surface of the negatively charged polysaccharide inner core is covered with a positively charged polysaccharide outer shell.

8. The method of fabricating the drug carrier according to claim 7, wherein a component of the negatively charged polysaccharide solution comprises alginate, gelatin, or a combination thereof.

9. The method of fabricating the drug carrier according to claim 7, wherein a component of the positively charged polysaccharide solution comprises chitosan.

10. The method of fabricating the drug carrier according to claim 7, wherein the boron-containing drugs comprise boric acid, boronophenylalanine, sodium borocaptate, or a combination thereof.

11. The method of fabricating the drug carrier according to claim 7, wherein the aqueous solution comprises a strontium chloride aqueous solution, a calcium chloride aqueous solution, or a combination thereof.

12. The method of fabricating the drug carrier according to claim 7, wherein an applied voltage range of the electrospray process is between 13 kV and 15 kV.

13. The method of fabricating the drug carrier according to claim 7, wherein a flow rate range of the mixed solution in the electrospray process is between 20 microliters/hour and 50 microliters/hour.

14. The method of fabricating the drug carrier according to claim 7, wherein a time for immersing the negatively charged polysaccharide inner core in the positively charged polysaccharide solution is 12 hours or more.

15. The method of fabricating the drug carrier according to claim 7, wherein a temperature for immersing the negatively charged polysaccharide inner core in the positively charged polysaccharide solution is room temperature.

16. The method of fabricating the drug carrier according to claim 7, wherein a particle size range of the drug carrier is between 150 nanometers and 250 nanometers.

17. The method of fabricating the drug carrier according to claim 7, wherein the positively charged polysaccharide outer shell covers the surface of the negatively charged polysaccharide inner core by electrostatic attraction.

18. The method of fabricating the drug carrier according to claim 7, wherein a loading efficiency range of the drug carriers to the boron-containing drugs is between 15% and 25%.
